(19) United States
(12) Patent Application Publication
(10) Pub. No.: US 2023/0002798 A1
(43) Pub. Date: Jan. 5, 2023

(54) PROCESS FOR THE PREPARATION OF A FERMENTABLE SUGAR COMPOSITION AND THE FERMENTATION THEREOF

(71) Applicants: WeissBioTech GmbH, Ascheberg (DE); BRAIN Biotech AG, Zwingenberg (DE)

(72) Inventors: Johannes DE BIE, Ascheberg (DE); Mareike DIRKS-HOFMEISTER, Ascheberg (DE); Alexander PELZER, Bickenbach (DE); Ludwig KLERMUND, Ascheberg (DE)

(73) Assignees: WeissBioTech GmbH, Ascheberg (DE); BRAIN Biotech AG, Zwingenberg (DE)

(21) Appl. No.: 17/784,904

(22) PCT Filed: Dec. 11, 2020
(86) PCT No.: PCT/EP2020/085744

§ 371 Date: Jun. 13, 2022

(30) Foreign Application Priority Data
Dec. 13, 2019 (EP) 19216155.2

(51) Int. Cl. C12P 19/16; C12N 9/24; C12P 19/04

(52) U.S. Cl. CPC C12P 19/16; C12N 9/2402; C12P 19/04; C12Y 302/01003; C12Y 302/0101

(57) ABSTRACT

The present invention relates to a process for the preparation of a fermentable sugar composition, wherein a polysaccharide composition is treated by at least one glucoamylase enzyme and at least one oligo-1,6-glucosidase enzyme, and wherein the fermentable sugars are removed during the process. Thereby, the otherwise non-fermentable sugars can be utilized in the fermentation process to yield a fermentation product such as an alcohol or an organic acid or amino acid.

### FIELD OF THE INVENTION

The invention relates to a process for the preparation of a fermentable sugar composition and the fermentation thereof.

### BACKGROUND OF THE INVENTION

Starch is one of the important polysaccharide sources of fermentable sugars. During starch processing, starch is hydrolysed to dextrins and further to fermentable sugars like glucose or maltose. Enzymatic aids for those hydrolytic processes are α-amylases (α-1,4-glucan-4-glucanohydrolases, E.C. 3.2.1.1) to degrade high-molecular starch comprising amylose and amylopectin to liquified polysaccharide comprising lower molecular weight dextrins (liquefaction process) and glucoamylase (1,4-α-D-glucan glucohydrolase, E.C. 3.2.1.3) to cleave the α-1,4 glycosidic linkages of those dextrins releasing glucose (saccharification process).

Such fermentable sugars are of high relevance for the food sector and can be further processed e.g. by yeast fermentation to bioethanol.

### SUMMARY OF THE INVENTION

Fermentation of starch can be achieved either by performing the saccharification and fermentation in subsequent or separate processes, or by combining the two steps into one single SSF-process standing for Simultaneous Saccharification and Fermentation (FIG. 1).

In an economically ideal case, the utilized liquefied polysaccharide substrate should be converted almost completely to the fermentation product with as little loss as possible. However, such losses regularly occur by the unwanted accumulation of non-fermentable sugars and thus give rise to economical and technical problems. Those non-fermentable sugars are oligosaccharides that are not linked via α-1,4-glycosidic linkages and are typically not accessible in the fermentation process.

The origins of these unwanted glycosides are (i) from the polysaccharide source by leftover α-1,6-linkages of amylopectin (α-limit dextrins), (ii) from the amylase enzyme as unwanted side-products after pro-longed liquefaction, (iii) from side-activities of the glucoamylase used in saccharification or (iv) from the metabolisms of the respective fermenting organisms. As a result, non α-1,4-linked glycosides, comprising panose (α-D-Glc-[1→6]-α-D-Glc-[1→4]-D-Glc), isomaltotriose (α-D-Glc-[1→6]-α-D-Glc-[1→6]-D-Glc), isomaltose (α-D-Glc-[1→6]-α-D-Glc) and/or trehalose (α-D-Glc-[1→1]-α-D-Glc), accumulate during the fermentation process. These accumulated non-fermentable sugars can constitute up to 3% of the total glycosides during the fermentation process. Hence there is a pressing need for a process utilizing alpha-1,4-linked glycosides as well as alpha-1,6-linked glycosides during a fermentation or SSF. This can be achieved by the method of the present invention.

It is an aspect of the present invention to utilize these non-fermentable sugars and thus transfer them from an economical loss into an increased yield.

It is a further aspect of the invention to use oligo-1,6-glucosidases (oligosaccharide α-1,6-glucosidase, E.C. 3.2.1.10) as a solution to the problems in the fermentation or SSF process described above.

It is a further aspect of the present invention to provide a process for the preparation of a fermentable sugar composition, wherein a polysaccharide composition is treated by at least one protein having glucoamylase enzymatic activity and at least one protein having oligo-1,6-glucosidase enzymatic activity, and wherein fermentable sugars are (substantially) removed during the treatment with the protein having oligo-1,6-glucosidase enzymatic activity.

### BRIEF DESCRIPTION OF THE DRAWINGS

FIG. 1: Scheme on processing starch to gain fermentation products. Starch is first treated with α-amylase enzymes for liquefaction to gain dextrins. Those are then either processed in an SSF (simultaneous saccharification and fermentation) containing mixtures of glucoamylase enzyme, an oligo-1,6-glucosidase as described in this invention and a fermenting organism, to yield a fermentation product. Alternatively, a separated process would first saccharify the dextrins with a glucoamylase enzyme to a syrup, which is subsequently used as substrate for a fermentation step in which an oligo-1,6-glucosidase enzyme is applied.

FIG. 2: Shows ethanol production (A) and degradation of panose (B) in SSF after 24 h. Samples contained maltodextrins and additional 2% panose. Negative control (neg.) contains glucoamylase only, +Agl1 and +Gth contain glucoamylase plus 187.5 kU per ton of sugar/starch equivalent of said oligo-1,6-glucosidase Agl1 or Gth, respectively. Positive control (pos.) contains additional 2% glucose instead of the panose and glucoamylase only.

FIG. 3: Shows ethanol production in SSF after 24 h from maltodextrins with additional 2% trehalose (A), or 2% isomaltose (B), or 2% isomaltotriose (C), or 2% maltose (D) or 2% maltotriose (E). Negative control (neg.) contains glucoamylase only, +Agl1 and +Gth contain glucoamylase plus 187.5 kU per ton of sugar/starch equivalent of said oligo-1,6-glucosidase Agl1 or Gth, respectively. In (A) 937.5 kU per ton of sugar/starch equivalent of said oligo-1,6-glucosidase Agl1 or Gth, respectively, were used. Positive control (pos.) contains additional 2% glucose instead of the respective sugar and glucoamylase only.

FIG. 4: Illustrates the course of ethanol concentration during SSF when comparing experiments with (o) or without (⋅) addition of a 187.5 kU per ton of sugar/starch equivalent of oligo-1,6-glucosidase (Agl1 or Gth). Samples contained maltodextrins and additional 2% panose.

FIG. 5: Shows the ethanol production in SSF with liquefied starch after 72 h. Negative control (neg.) contains glucoamylase only, +Agl1 and +Gth contain glucoamylase plus 187.5 kU per ton of sugar/starch equivalent of said oligo-1,6-glucosidase Agl1 or Gth, respectively

FIG. 6: Shows overlay of HPLC chromatograms of the liquefied starch prior to SSF (black) and a standard (dotted) containing 5 g/L of glucose^{\textcircled{1}}, maltose^{\textcircled{2}}, isomaltose^{\textcircled{3}}, maltotriose^{\textcircled{4}}, panose^{\textcircled{5}}, isomaltotriose^{\textcircled{6}}. The liquefied starch contains no detectable non-fermentable sugars. R means refractive index (of HPLC efflux).

FIG. 7: Shows HPLC chromatograms (dotted lines) showing residual sugars after SSF without oligo-1,6-glucosidase (A), with Agl1 (B), and with Gth (C) and in comparison a standard (black) containing 5 g/L of glucose^{\textcircled{1}}, maltose^{\textcircled{2}}, maltotriose^{\textcircled{4}}, panose^{\textcircled{5}}, isomaltose^{\textcircled{3}}, isomaltotriose^{\textcircled{6}}. After SSF, no non-fermentable sugars (panose, isomaltose, isomaltotriose, trehalose) were detected. R means refractive index (of HPLC efflux).

FIG. 8: Shows panose concentration during SSF with liquefied starch measured after 66 h and 72 h. Negative control (neg.) contains glucoamylase only, +Agl1 and +Gth contain glucoamylase plus 187.5 kU per ton of sugar/starch equivalent of said oligo-1,6-glucosidase Agl1 or Gth, respectively.

FIG. 9: Illustrates the biochemical characterization of Agl1. Activity is shown for different sugars at 10 mM and 50 mM. Herein, the term “Units” (U) refers to the amount of enzyme required to produce one μmol glucose per minute. Error bars are standard deviations of at least triplicates.

FIG. 10: Illustrates the biochemical characterization of Gth. Activity is shown for different sugars at 100 mM, 50 mM, or 10 mM and for panose at 15 mM. Units refers to the amount of enzyme required to produce one μmol glucose per minute. Error bars are standard deviations of at least triplicates.

FIG. 11: Illustrates the product inhibition of Agl1 by various glucose concentrations (A) and various maltose concentrations (B). The residual activity of Agl1 at various product concentrations is relative to the activity without additional product.

### DETAILED DESCRIPTION OF THE EMBODIMENTS

In one aspect the invention relates to an SSF process for the preparation of a fermentable sugar composition, wherein a polysaccharide composition is treated by an enzyme mixture of at least one protein having glucoamylase enzymatic activity and at least one protein having oligo-1,6-glucosidase enzymatic activity, and wherein the fermentable sugars are (substantially) removed during the process.

As indicated above, in embodiments the fermentable sugars are (substantially) removed during the treatment with the protein having oligo-1,6-glucosidase enzymatic activity. The removal may especially be done in order to prevent inhibition of the enzymes with oligo-1,6-glucosidase activity. Hence, in embodiments during the process for the preparation of a fermentable sugar composition the fermentable sugars may (substantially) be removed. Therefore, in specific embodiments there may essentially be no detectable amounts of fermentable sugars.

According to a further aspect of the invention the polysaccharide composition is first treated by at least one protein having glucoamylase enzymatic activity and the resulting composition is subsequently treated with at least one protein having oligo-1,6-glucosidase enzymatic activity and wherein the fermentable sugars are (substantially) removed during the treatment with the protein having oligo-1,6-glucosidase enzymatic activity According to one embodiment of the present invention the fermentable sugars are removed by consumption thereof in a fermentation process.

In the present context with “fermentation process” we mean a metabolic process that changes the biochemical composition of an organic medium through the action of metabolising microorganisms. During said process parts of the organic medium (e.g. sugars, proteins, nutrients) are consumed by the microorganisms to produce biomass, carbon dioxide and other fermentation products.

According to one embodiment of the present invention the fermentation process may comprise producing a fermentation product. Hence, in embodiments, the fermentable sugar composition may be fermented to yield a fermentation product. The fermentation product may comprise any molecule producible by a (fermenting) microorganism. In further embodiments, the fermentation product may comprise one or more of an alcohol, an organic acid, or an amino acid.

According to one embodiment of the present invention the fermentable sugar composition is fermented to yield an alcohol. For such alcoholic fermentation, Saccharomyces cerevisiae is the most commonly employed yeast in industrial ethanol production, combining high ethanol productivity and tolerance (18% v/v) and growth in low pH environments (reducing bacterial contamination). However, any microorganism capable of alcoholic fermentation can be deployed, such as different yeasts (e.g. Hanseniaspora, Candida, Cyberlindnera, Kluyveromyces, Zygosaccharomyces, Schizosaccharomyces, Torulaspora, Dekkera Brettanomyces), bacteria (Microbacterium, Corynebacterium, Gluconobacter, Acetobacter, Pseudomonas, Vibrio, Gliocladium, Lactobacillus, Erwinia, Bacillus, Brevibacterium, Arthrobacter) and fungi (e.g. Aspergillus, Penicillium, Rhizopus).

According to a further aspect the present invention relates to a process for the fermentative production of an alcohol wherein a polysaccharide composition is treated by an enzyme mixture of at least one glucoamylase enzyme and at least one oligo-1,6-glucosidase enzyme, thereby providing a fermentable sugar composition, wherein the fermentable sugar is simultaneously fermented to yield the alcohol.

According to a further embodiment of the present invention the fermentable sugar composition is fermented to yield organic acids or amino acids.

Organic acids which can be prepared by fermentation are for example citric acid, gluconic acid, malic acid, lactic acid, acetic acid, L-ascorbic acid, itaconic acid, propionic acid, succinic acid and/or pyruvic acid.

Citric acid typically can be prepared by Aspergillus sp., Penicillium sp., Candida sp., Ustilago sp. or Corynebacterium sp. Gluconic acid typically can be prepared by Gluconobacter, Acetobacter, Pseudomonas, Vibrio, Aspergillus, Penicillium, Ustilago or Gliocladium. Lactic acid typically can be prepared by Lactobacillus. Acetic acid typically can be prepared by Gluconobacter or Acetobacter. L-ascorbic acid typically can be prepared by Acetobacter, Erwinia, Bacillus or Corynebacterium. Itaconic acid and malic acid can for example be prepared by Aspergillus or Ustilago.

According to a further aspect the present invention relates to a process for the fermentative production of an organic acid wherein a polysaccharide composition is treated by an enzyme mixture of at least one glucoamylase enzyme and at least one oligo-1,6-glucosidase enzyme, thereby providing a fermentable sugar composition, wherein the fermentable sugar is simultaneously fermented to yield the organic acid.

Amino acids which can be prepared by fermentation are for example alanine, arginine, glutamic acid, glutamine, lysine, proline, threonine, tryptophan, valine.

Alanine can for example be prepared by Microbacterium ammoniaphilum. Arginine can suitably be produced by certain Serratia marcescens species. Glutamic acid can be produced by Corynebacterium glutamicum, Brevibacterium flavum or Arthrobacter paraffineus. Glutamine can be produced by Corynebacterium glutamicum. Lysine can be prepared by Brevibacterium lactofermentum or Brevibacterium flavum. Proline can be produced by Corynebacterium acetocidophylum. Threonine as well as tryptophan can be produced by certain E. coli species. Valine can be produced by certain Brevibacterium lactofermentum species.

According to a further aspect the present invention relates to a process for the fermentative production of an amino acid wherein a polysaccharide composition is treated by an enzyme mixture of at least one glucoamylase enzyme and at least one oligo-1,6-glucosidase enzyme, thereby providing a fermentable sugar composition, wherein the fermentable sugar is simultaneously fermented to yield the amino acid.

In a further embodiment, the present invention also relates to a process for the production of a fermentation product from a polysaccharide composition, wherein the polysaccharide composition is treated by at least one protein having glucoamylase enzymatic activity and at least one protein having oligo-1,6-glucosidase enzymatic activity, and wherein the resulting fermentable sugar composition is (substantially) removed during the treatment with the protein having oligo-1,6-glucosidase enzymatic activity by a fermentation process in a fermentation medium, and wherein the desired fermentation product is isolated from the fermentation medium.

According to a particular embodiment, the fermentation product (such as an alcohol, an organic acid or an amino acid) is isolated from the fermentation medium in an aqueous medium.

For the isolation of the fermentation product (optionally after removal of all particulate material) any method known in the art for these products may be applied, such as chromatography and (in particular for lower alcohols) distillation.

In the present context a fermentable sugar composition comprises glucose, maltose and other α-1,4-linked oligo-saccharides (maltodextrins).

The polysaccharide composition which can be used according to the present invention is any composition comprising maltodextrins or other dextrins, and can for example be a starch hydrolysate. Preferably a starch hydrolysate can be used, which can be obtained from starch by acid-treatment or enzymatic digestion e.g. with α-amylase. The starch may be derived from any of various plant sources (such as corn, wheat, potato, rice and cassava).

In the present context a protein having glucoamylase enzymatic activity may mean a native glucoamylase derived from a natural source or a variant or mutant protein having at least 50%, such as at least 55%, or at least 60%, or at least 65%, or at least 70%, or at least 75%, or at least 80%, or at least 85%, or at least 90%, or at least 95% %, or at least 98% sequence identity to a glucoamylase derived from a natural source, while still having glucoamylase enzymatic activity.

For use in the fermentation processes according to the present invention a glucoamylase of the enzyme class EC 3.2.1.3 can be selected from diverse microbiological sources, mostly moulds and yeasts but also bacteria. Examples of sources for industrial use are Aspergillus sp., Rhizopus sp. and Bacillus sp. It is known to hydrolyse terminal (1→4)-linked alpha-D-glucose residues successively from non-reducing ends of polysaccharide chains with release of beta-D-glucose. The systematic name of this enzyme is glucan 1,4-alpha-glucosidase. Equivalent names for this enzyme may include amyloglucosidase (AMG), gamma-amylase, lysosomal alpha-glucosidase, acid maltase, exo-1,4-alpha-glucosidase, glucose amylase, gamma-1,4-glucan glucohydrolase, acid maltase, 1,4-alpha-D-glucan glucohydrolase, 4-alpha-D-glucan glucohydrolase.

In the present context a protein having oligo-1,6-glucosidase enzymatic activity may mean a native oligo-1,6-glucosidase derived from a natural source or a variant or mutant protein having at least 50%, such as at least 55%, or at least 60%, or at least 65%, or at least 70%, or at least 75%, or at least 80%, or at least 85%, or at least 90%, or at least 95% %, or at least 98% sequence identity to a oligo-1,6-glucosidase derived from a natural source, while still having oligo-1,6-glucosidase enzymatic activity.

For use in the fermentation processes according to the present invention an oligo-1,6-glucosidase of the enzyme class EC 3.2.1.10 may be selected from diverse sources, like e.g. from Bacillus sp., Bifidobacterium sp., Saccharomyces sp., Sulfolobus sp., but also from higher organisms like human or plants. It is known to hydrolyse (1→6)-alpha-D-glucosidic linkages in oligosaccharides. The systematic name of this enzyme is oligosaccharide 6-alpha-glucohydrolase, and this enzyme is also known as oligo-1,6-glucosidase, alpha-glucosidase, dextrin 6-alpha-D-glucanohydrolase, disaccharidase, exo-oligo-1,6-glucosidase, sucrase, isomaltase, limit-dextrinase, oligosaccharide alpha-1,6-glucosidase, sucrase isomaltase.

Several oligo-1,6-glucosidase sequences are publicly available, however neither their performance under process conditions (pH 4.3-4.8, 30-35° C. for fermentative alcohol production, concomitance with living microorganisms and high sugar contents) nor any effect of their use in fermentation processes were shown.

In particular, a process according to the present invention may apply an enzyme based on oligo-1,6-glucosidase Gth of Parageobacillus thermoglucosidasius as described in WO2009/152285 and U.S. Pat. No. 9,127,287 or on oligo-1,6-glucosidase Agl1 of Bifidobacterium breve UCC2003 as described in U.S. Pat. No. 7,268,221B2 and U.S. Pat. No. 7,615,365B2.

This successful performance of the process according to the invention is particularly surprising because (i) the amount of non-fermentable sugars is very low resulting in low enzyme activity, (ii) the content of fermentable sugars and thus products of the oligo-1,6-glucosidase reaction can be very high and thus may lead to product inhibition, and (iii) also 1,1-linked non-fermentable sugars (Trehalose) can be converted into fermentable sugar and leads to an increase in the yield of fermentation product. Most surprisingly, the effect on the sugar profile and fermentative production of the SSF surpasses that of the expected content of non-fermentable sugars. This is illustrated in Example 3 wherein we find an increase in fermentation product although the control without oligo-1,6-glucosidase has no detectable amounts of non-fermentable sugars.

Our experiments under application conditions surprisingly showed that any of the oligo-1,6-glucosidases of Bifidobacterium breve UCC2003 (Agl1; amino acid sequence SEQ ID NO:1) and Parageobacillus thermoglucosidasius (Gth; amino acid sequence SEQ ID NO:2) used in cooperation with glucoamylase enzyme resulted in a substantial increase of fermentation yields.

For use according to the present invention the protein having oligo-1,6-glucosidase enzymatic activity may comprise a protein having at least 50%, such as at least 55%, or at least 60%, or at least 65%, or at least 70%, or at least 75%, or at least 80%, or at least 85%, or at least 90%, or at least 95% %, or at least 98% sequence identity to the amino acid sequence SEQ ID NO:1 of oligo-1,6-glucosidase Agl1 of Bifidobacterium breve UCC2003 or to the amino acid sequence SEQ ID NO:2 of oligo-1,6-glucosidase Gth of Parageobacillus thermoglucosidasius, while still having oligo-1,6-glucosidase enzymatic activity.

It has been found that Agl1 and Gth addition shows a 2% increase of ethanol yield from a lab-scale SSF run with 32% dextrins supplemented with panose, 30° C. at pH 4.8 for 72 h with commercial glucoamylase (415 g/t of starch Deltazym® GA L-E5) and alcohol yeasts (6 g/L fermentation Deltaferm® AL-18).

An enzyme used in the process according to the present invention may be obtained from the organism which naturally produces such enzyme. Alternatively, the enzyme may be produced by a host cell which is transformed to produce the enzyme or several of the enzymes needed in the process of the invention by recombinant techniques known in the art.

In one embodiment an enzyme may be applied during the process of the invention in the form of whole cells producing the enzyme.

In a further embodiment, an enzyme may be applied during the process of the invention in the form of a lysate of the cells producing the enzyme.

In a further embodiment, an enzyme may be applied during the process of the invention in a more or less purified form, such as in a form essentially free from particulate material from the producing cells. Purification of the enzyme may be performed by any method known in the art.

In a further embodiment, an enzyme may be applied during the process of the invention in immobilized form. Many useful methods for immobilization of enzymes are known in the art. The enzymes may be immobilized for example by covalent bonding to a suitable surface or a carrier, by adsorption to a suitable surface, by entrapment e.g. in microspheres, by cross-linking e.g. to each other and/or with a suitable matrix material, or by affinity binding.

The enzymes glucoamylase and oligo-1,6-glucosidase may be used in the process of the invention in a concentration chosen to be optimal for the enzymatic conversions, and the concentration may range between 10 and 1000 kU per ton of dry cereal, preferentially between 50 and 200 kU per ton of dry cereal.

Given the nature of the polysaccharide composition used as a starting material for the process according to the invention the process will generally take place in an aqueous process medium, preferably in water. However, the process medium may also contain any aqueous buffer, e.g. phosphate buffer, bicarbonate buffer, or any other buffer solution, or mixtures of buffer solutions, or unbuffered solutions, or containing organic solvents, e.g. ethanol, methanol, DMSO, or any organic solvent, or combinations of organic solvents.

During the process of the invention the pH of the medium can vary from 4.0 to 8.0, with an optimal range of pH 4.0 to 5.5, more specifically of pH 4.3 to 4.8, and may also be dependent on the nature and concentration of the product produced by the fermentation.

During the process of the invention the temperature may range between 20 to 70° C., with an optimal range of 30 to 50° C., more specifically of 30 to 35° C.

It will be clear to the person skilled in the art that the process conditions may be selected based on the nature and concentration of the fermentation product and on the fermenting organism. Hence, the process conditions may especially be selected to be suitable for the fermenting organism to produce the (desired) fermentation product.

### EXAMPLES

### General Methods

Cloning and Expression

For use of Agl1 and Gth during the processes described in the invention, the enzymes need to be applied in their enzymatic active form. A person skilled in the art will have the ability to use a suitable expression system consisting of a suitable expression host, such as a microorganism, e.g. Bacillus subtilis, and a suitable expression construct fitting the expression host, to produce active enzymes.

The genes of Agl1 and Gth were obtained from public databases (National Center for Biotechnology Information (NCBI), US) [Agl1: GenBank FJ386389.1; Gth: GenBank NZ_CP012712.1 Region: 1287657-1289345] and cloned into a suitable Bacillus-expression vector known in the art, such as derivates of pUB110 (American Type Culture Collection ATCC© 37015™, Zyprian & Matzura 1986) or pMA5 (Dartois et al. 1994), containing a promotor sequence, such as Pveg, PhpaII, PamyE or P43, upstream of the gene and a termination sequence, such as rrnB, downstream of the gene. If using such common shuttle vectors an origin of replication for propagation in Escherichia coli and an origin of replication for replication in Bacillus subtilis as well as selection markers, such as ampicillin or kanamycin, need to be present. In the present invention, such a vector was constructed and propagated in E. coli prior to transformation into B. subtilis as exemplary host for enzyme production.

For production of Agl1 and Gth, Bacillus subtilis was grown overnight in LB medium as a liquid culture. One millilitre of the liquid culture was used as inoculum for expression in 100 mL A5 low phosphate medium (1.76 g/L K₂HPO₄, 2 g/L (NH₄)₂SO₄, 3.63 g/L Na₂HPO₄, 10 g/L glucose, 1.23 g/L MgSO4*7H₂O, 10 g/L casamino acids, pH 6.8) supplemented with trace elements (50 mM FeSO₄, 10 mM MnSO₄, 10 mM ZnSO₄, 2 mM CoSO₄, 2 mM CuSO₄, 2 mM NiSO₄, 2 mM Na₂MoO₄, 2 mM Na₂SeO₃, 2 mM H₃BO₃). After 16 h constitutive expression, the cells were harvested by centrifugation at 8000×g and resuspended in 20 mM phosphate buffer, pH 7.4. The cells were broken by ultrasonication in a Branson sonifier (duty cycle 50% output control 7-9, 2 min, up to 5 repeats). The enzyme product was either used as crude sterile-filtered Bacillus extract, or centrifuged at 17,000×g to remove cell debris and purified using anion exchange chromatography (XK-16 column, IEX-Q-Beads, Bio-Rad Laboratories, Inc., US) followed by size exclusion chromatography (Superdex 200 Increase 10/300 GL, GE Healthcare).

Determination of Protein Amount

The amount of enzyme product (Agl1 and Gth respectively) was determined via densitometry on a standard SDS-PAGE and via activity measurements. Whole protein content and purified enzyme concentration were determined via BCA assay (Pierce, Thermo Fischer Scientific).

Activity Measurements

Glucosidase activity was quantified via a glucose assay kit (D-Glucose HK Assay Kit, Megazyme u.c. IRE). Panose was added to the enzyme product at 15 mM in 50 mM Britton-Robinson buffer, pH 5. The reaction was allowed to proceed for 10 min at 37° C. The reaction was stopped by the addition of 60 μL 1 M NaOH. The pH was neutralized by the addition of 60 μL 1 M HCl. The produced glucose was quantified using the glucose assay kit following the manufacturer's instructions. One Unit (U) refers to the amount of enzyme needed to produce 1 μmol of glucose per minute.

Quantification of ethanol yield Ethanol was quantified via HPLC. Samples were taken at the end of the SSF or during SSF at set time points. Samples were centrifuged for 1 min at 17,000 xg and sterile filtered (0.2 μm). The samples were frozen at −80° C. prior to measurements. Ethanol was determined with an Aminex 87C column (Bio-Rad Laboratories, Inc., US) on a Shimadzu HPLC system.

Quantification of Residual Sugars

Residual sugars were quantified via HPLC. Samples were taken at the end of the SSF or during SSF at set time points. Samples were centrifuged for 1 min at 17 000 xg and sterile filtered (0.2 μm). The samples were frozen at −80° C. prior to measurements. Residual sugars were determined with a Zorbax NH2 column (Agilent Technologies, Inc., US) on an Agilent 1200 HPLC system.

Analysis of Liquefied Starch

Liquefied starch was analysed prior to use on a Zorbax NH2 column (Agilent Technologies, Inc., US) for analysis of mono-, di- and trisaccharide type and content, and on an Aminex 42A column (Bio-Rad Laboratories, Inc., US) for analysis of degree of polymerisation (DP) detecting DP1-DP6 content. IPLC was performed on a Shimadzu IPLC system.

### Example 1

Conversion of non-fermentable sugars to ethanol to test functionality of oligo-1,6-glucosidases in SSF set-up

Simultaneous Saccharification and Fermentation

For simultaneous saccharification and fermentation (abbreviated SSF), the oligo-1,6-glucosidases—either Agl1 or Gth—were used as crude Bacillus extract. Glucidex® Maltodextrin 12 (Roquette, FR) was dissolved in SSF-medium (5 g/L yeast extract, 5 g/L peptone, 1 g/L K₂HIPO₄, 0.5 g/L Mg₂SO₄ in 50 mM citrate buffer, pH 4.8) at 300 g/L. Panose, isomaltose, isomaltotriose, maltose, maltotriose, or trehalose was added at 20 g/L. As a positive control, 20 g/L glucose was added instead of the respective sugar. The SSF medium containing starch hydrolysate (maltodextrins) and sugars was sterile filtered (0.2 μm). Yeast (Deltaferm® AL-18, WeissBioTech GmbH, Ascheberg, Germany) was grown in YINaC medium (50 g/L dextrose, 5 g/L yeast extract, 5 g/L peptone, 1 g/L K₂HPO₄, 0.5 g/L Mg₂SO₄*7H₂O, 2 g/L NH₄Cl in 50 mM sodium citrate, pH 4.8) for 2 days at 37° C. and harvested by centrifugation for 15 min at 5100 rpm and 8° C. Pelleted yeast was added to the SSF-medium at a final concentration of 6 g/L. Glucoamylase (Deltazym® GA L-E5, WeissBioTech GmbH, Ascheberg, Germany) was added at 40 g per ton of dry starch equivalent. The enzyme product Agl1 or Gth was added up to a final concentration of 187.5 kU per ton of dry starch equivalent. For the SSF with trehalose, Agl1 or Gth was added at 937.5 kU per ton of dry starch equivalent. As a negative control, an SSF as described above was run without oligo-1,6-glucosidase/enzyme product. The SSF was performed at 30° C. without stirring or shaking for 24 h.

Results:

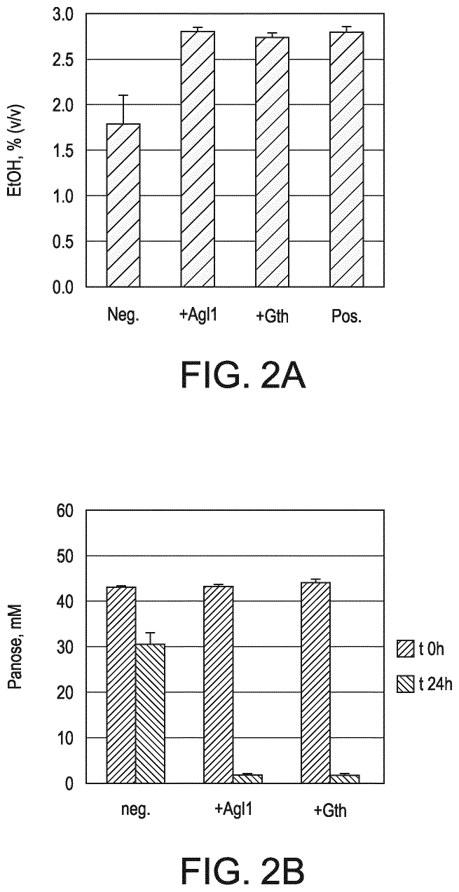

With the addition of panose, ethanol concentration after 24 h increased by 1.01 percentage points (v/v, increase by 92%) with Agl1 and 0.94% percentage points (v/v, increase by 85%) with Gth compared to the negative control (FIG. 2A). The ethanol yield was not significantly different from the positive control (−0.99 percentage points v/v; 90%). Panose concentration was reduced by 96% with Agl1, by 96% with Gth and in the negative control by 29% with glucoamylase only (FIG. 2B).

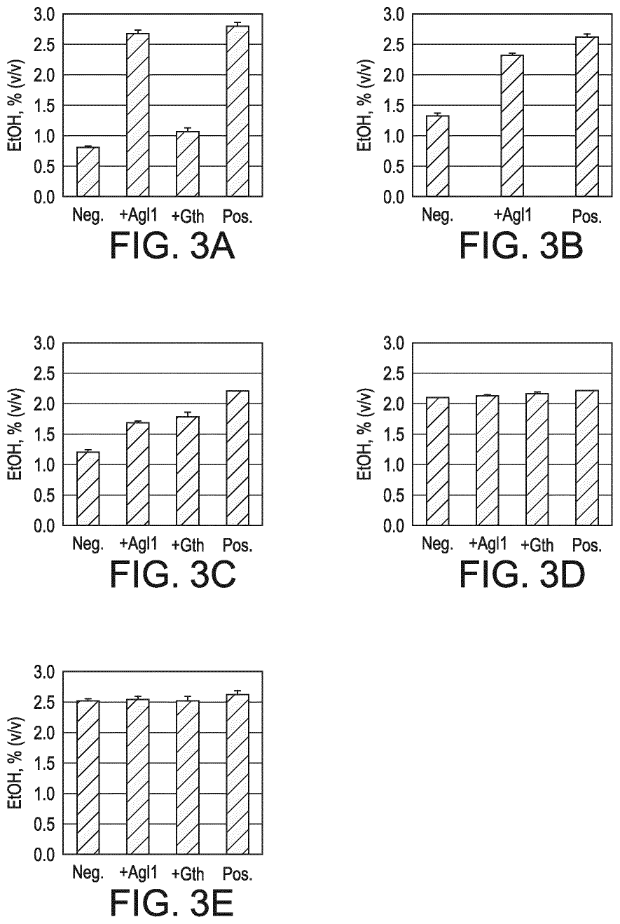

With the addition of trehalose, ethanol concentration after 24 h increased by 1.86 percentage points (v/v, increase by 226%) with Agl1 and 0.26 percentage points (v/v, increase by 31%) with Gth compared to the negative control (FIG. 3A). The ethanol yield in the positive control was 1.95 percentage points (v/v) higher compared to the negative control (increase by 238%).

With the addition of isomaltose, ethanol concentration after 24 h increased by 0.98 percentage points (v/v, increase by 74%) with Agl1 compared to the negative control (FIG. 3B).

The ethanol yield was not significantly different from the positive control.

With the addition of isomaltotriose, ethanol concentration after 24 h increased by 0.48 percentage points (v/v, increase by 40%) with Agl1 and 0.57 percentage points (v/v, increase by 48%) with Gth compared to the negative control (FIG. 3C). The ethanol yield in the positive control was 1.00 percentage point (v/v) higher compared to the negative control (increase by 83%).

No increase in the ethanol yield was determined when maltose or maltotriose was added (FIG. 3D, E). With the addition of maltose, the ethanol concentration after 24 h increased by 0.03 percentage points (v/v, increase by 1.5%) with Agl1 and 0.06 percentage points (v/v, increase by 2.8%) with Gth compared to the negative control. The ethanol yield in the positive control was 0.11 percentage points (v/v) higher compared to the negative control (increase by 5.0%). With the addition of maltotriose, ethanol concentration after 24 h increased by 0.03 percentage points (v/v, increase by 1%) with Agl1 and 0.00 percentage points (v/v, increase by 0%) with Gth compared to the negative control. The ethanol yield in the positive control was 0.10 percentage points (v/v) higher compared to the negative control (increase by 4.0%).

Conclusion:

An increase in ethanol yield compared to the control without oligo-1,6-glucosidase was observed for the non-fermentable sugars panose, isomaltose, isomaltotriose and trehalose in an SSF set-up indicating full function of the enzyme products with regard to converting non-fermentable sugars to glucose in an SSF with glucoamylase and fermenting organism. The yields obtained by adding the fermentable sugars maltose and maltotriose are unaffected by oligo-1,6-glucosidase.

### Example 2

Conversion of panose to ethanol to test functionality of oligo-1,6-glucosidases in industry-relevant SSF set-up

Simultaneous Saccharification and Fermentation

For the SSF, the enzyme product was used as crude Bacillus extract. Glucidex® Maltodextrin 12 was dissolved in SSF medium (5 g/L yeast extract, 5 g/L peptone, 1 g/L K₂HIPO₄, 0.5 g/L Mg₂SO₄ in 50 mM citrate buffer, pH 4.8) at 300 g/L. Panose was added at 20 g/L. As a positive control, 20 g/L glucose was added. The SSF medium containing starch and sugars was sterile filtered (0.2 μm). Yeast (Deltaferm® AL-18, WeissBioTech GmbH, Ascheberg, Germany) was grown in YINaC medium (50 g/L dextrose, 5 g/L yeast extract, 5 g/L peptone, 1 g/L K₂HPO₄, 0.5 g/L Mg₂SO₄*7H₂O, 2 g/L NH₄Cl in 50 mM sodium citrate, pH 4.8) for 2 days at 37° C. and harvested by centrifugation for 15 min at 5100 rpm and 8° C. Pelleted yeast was added to the SSF at a final concentration of 6 g/L. Glucoamylase (Deltazym® GA L-E5, WeissBioTech GmbH, Ascheberg, Germany) was added at 400 g per ton of dry starch equivalent. The enzyme product Agl1 or Gth was added to a final concentration of 187.5 kU per ton of dry starch equivalent. As a negative control, no oligo-1,6-glucosidase/enzyme product was added. The SSF was performed at 30° C. without stirring or shaking for 72 h.

Results:

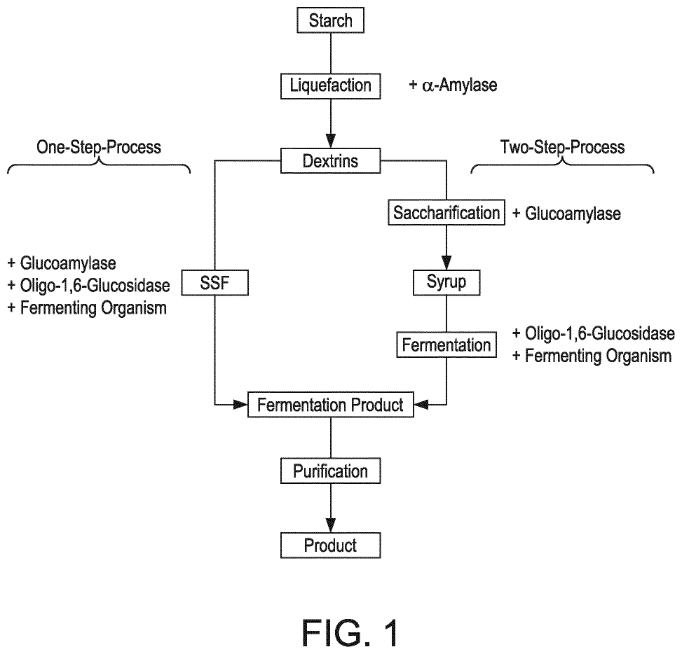

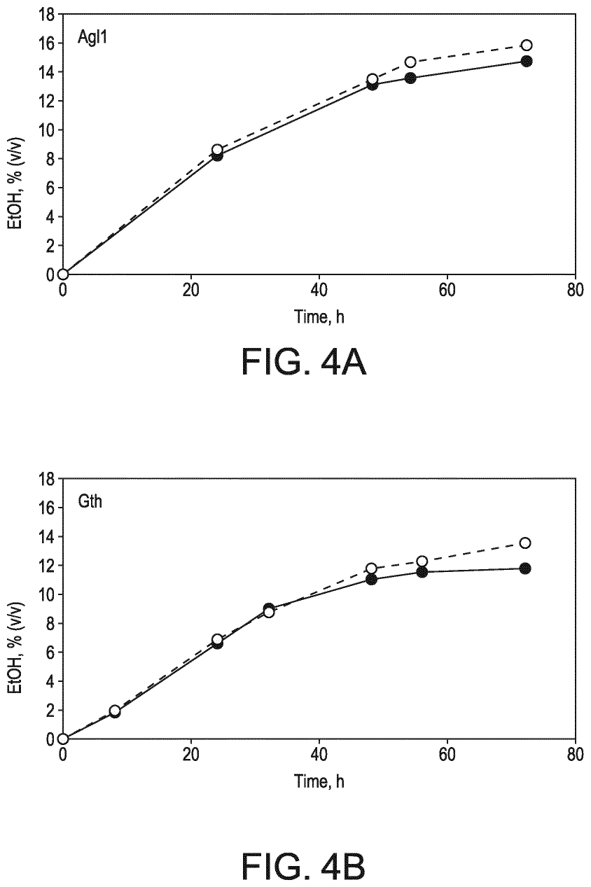

With the addition of panose, ethanol concentration after 72 h increased by 1.16 percentage points (v/v, increase by 8%) with Agl1 (FIG. 4A) and 1.77 percentage points (v/v, increase by 15%) with Gth (FIG. 4B) compared to the negative control. The ethanol yield was not significantly different from the positive control.

Conclusion:

An increase in ethanol yield compared to the control without oligo-1,6-glucosidase was observed for the non-fermentable sugar panose in a SSF with a process time of 72 h with the product recommended dose of glucoamylase and fermenting organism. Although the glucoamylase is capable of hydrolysing the respective amount of panose, a marked positive effect of the oligo-1,6-glucosidases Agl1 and Gth on the ethanol yield was detected. The oligo-1,6-glucosidases are stable and active for 72 h at pH 4.8 and 30° C.

### Example 3

Ethanol yield increase from commercial liquefied starch to show functionality with industry-relevant substrate in industry-relevant process conditions

Simultaneous Saccharification and Fermentation

For SSF, the enzyme product was used as crude Bacillus extract. Acidified liquefied starch (pH 2.0) from a commercial plant, containing no detectable amounts of panose, isomaltose, isomaltotriose or trehalose, was brought to pH 4.8. SSF medium components (5 g/L yeast extract, 5 g/L peptone, 1 g/L K₂HIPO₄, 0.5 g/L Mg₂SO₄ in 50 mM citrate buffer, pH 4.8) were added to liquefied starch (300 g/L). The SSF medium containing starch was sterile filtered (0.2 μm). Yeast (Deltaferm® AL-18, WeissBioTech GmbH, Ascheberg, Germany) was grown in YINaC medium (50 g/L dextrose, 5 g/L yeast extract, 5 g/L peptone, 1 g/L K₂HPO₄, 0.5 g/L Mg₂SO₄*7H₂O, 2 g/L NH₄Cl in 50 mM sodium citrate, pH 4.8) for 2 days at 37° C. and harvested by centrifugation for 15 min at 5100 rpm and 8° C. Pelleted yeast was added to the SSF at a final concentration of 6 g/L. Glucoamylase (Deltazym® GA L-E10, WeissBioTech GmbH, Ascheberg, Germany) was added at 200 g per ton of dry starch equivalent. The enzyme product Agl1 or Gth was added to a final concentration of 187.5 kU per ton of dry starch equivalent. As control, no oligo-1,6-glucosidase/enzyme product was added. The SSF was performed at 30° C. without stirring or shaking for 72 h.

Results:

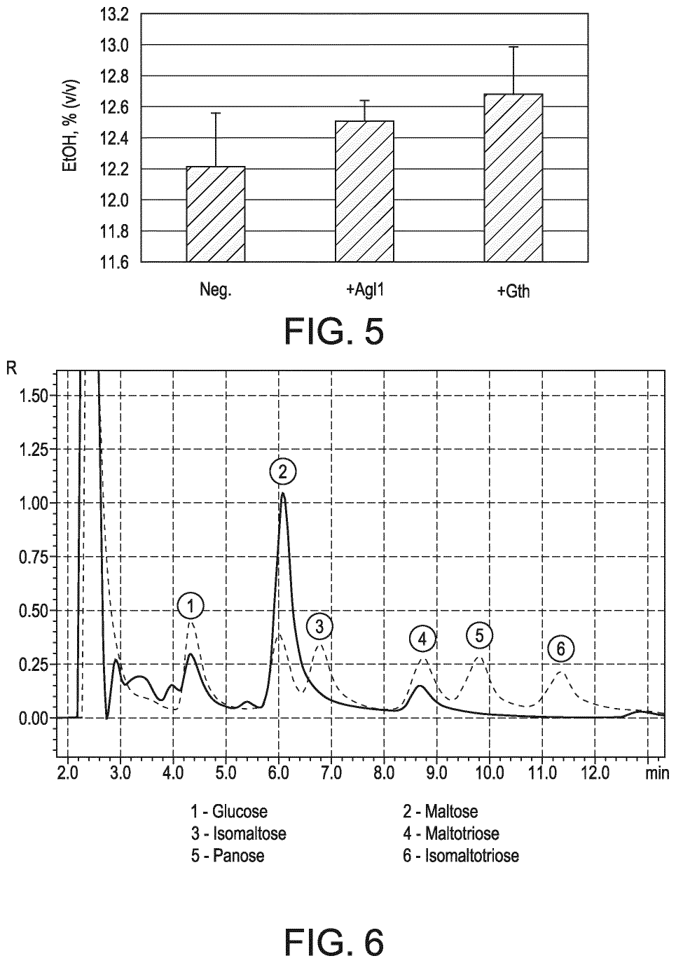

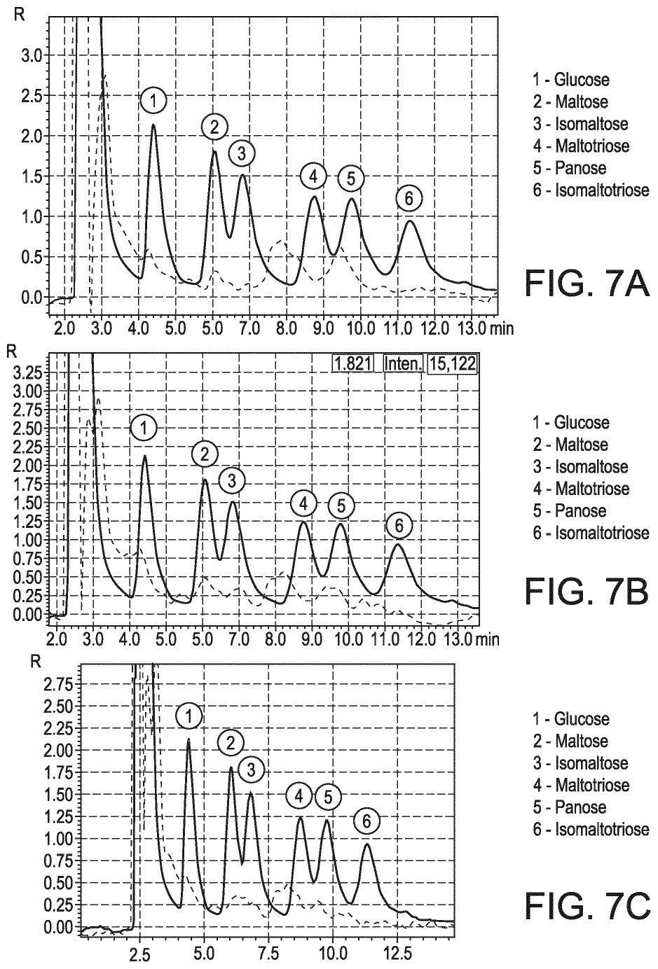

Ethanol concentration after 72 h increased by 0.30 percentage points (v/v, increase by 2%) with Agl1 and 0.47 percentage points (v/v, increase by 4%) with Gth compared to the negative control without oligo-1,6-glucosidase (FIG. 5). No non-fermentable sugars were detected in the liquefied starch prior to its use in SSF (FIG. 6). Interestingly, no residual sugars, neither fermentable nor non-fermentable and neither in the control nor in the SSFs containing oligo-1,6-glucosidase, were detected after SSF (exemplified in the control, FIG. 7). To the reader skilled in the art, a 2% ethanol yield increase during SSF is substantial.

Conclusion:

An increase in ethanol yield was observed when adding oligo-1,6-glucosidases to an SSF compared to the control without oligo-1,6-glucosidase after saccharification and fermentation of commercial liquefied starch under industry-relevant conditions.

Interestingly, no non-fermentable sugars were detected prior and after SSF in any of the performed SSFs. Especially, no residual sugars were detected in the SSF without oligo-1,6-glucosidases, demonstrating that the addition of the enzyme product has a positive effect on the SSF even without detectable residual sugars after SSF.

### Example 4

Reduction in DP3 (panose) sugar content after SSF with commercial liquefied starch to show functionality with industry-relevant substrate in industry-relevant process conditions

Simultaneous Saccharification and Fermentation

For SSF, the enzyme product was used as crude Bacillus extract. Acidified liquefied starch from a commercial plant, containing no detectable amounts of panose, isomaltose, isomaltotriose or trehalose, was brought to pH 4.8. SSF medium components (5 g/L yeast extract, 5 g/L peptone, 1 g/L K₂HPO₄, 0.5 g/L Mg₂SO₄ in 50 mM citrate buffer, pH 4.8) were added to liquefied starch (300 g/L). The SSF medium containing starch was sterile filtered (0.2 μm). Yeast (Deltaferm® AL-18, WeissBioTech GmbH, Ascheberg, Germany) was grown in YINaC medium (50 g/L dextrose, 5 g/L yeast extract, 5 g/L peptone, 1 g/L K₂HIPO₄, 0.5 g/L Mg₂SO₄*7H₂O, 2 g/L NH₄Cl in 50 mM sodium citrate, pH 4.8) for 2 days at 37° C. and harvested by centrifugation for 15 min at 5100 rpm and 8° C. Pelleted yeast was added to the SSF at a final concentration of 6 g/L. Glucoamylase (Deltazym® GA L-E10, WeissBioTech GmbH, Ascheberg, Germany) was added at 200 g per ton of dry starch equivalent. Alpha-amylase (Fuelzyme®, BASF) was added at 200 g per ton of dry starch equivalent to simulate non-complete removal of alpha-amylase in industrial processes, which is often the cause for increased non-fermentable sugars. The enzyme product Agl1 or Gth was added to a final concentration of 187.5 kU per ton of dry starch equivalent. As control, no oligo-1,6-glucosidase/enzyme product was added. The SSF was performed at 30° C. without stirring or shaking for 72 h.

Results:

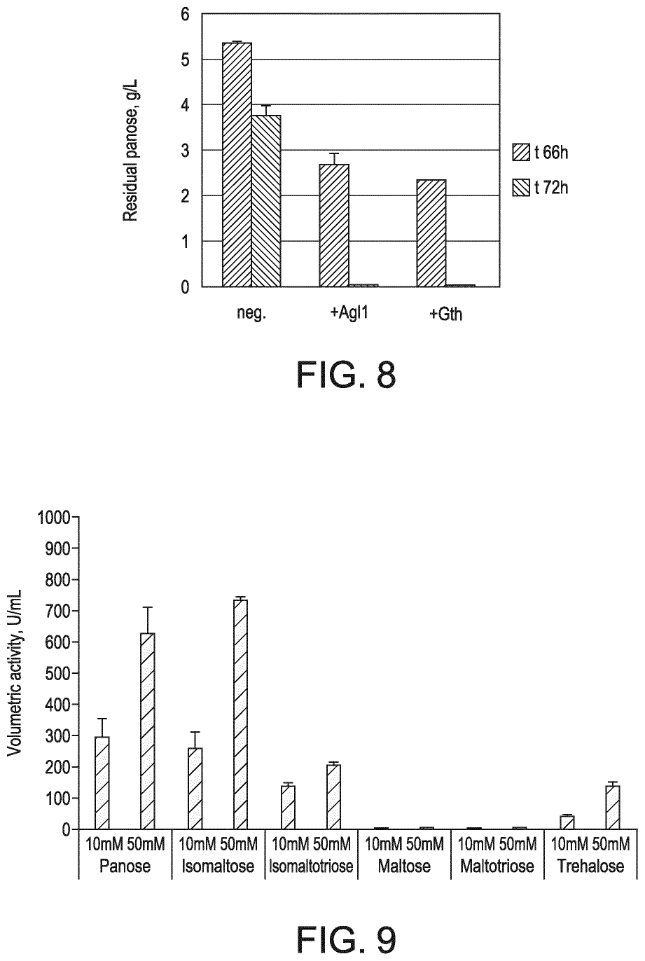

Panose was detected after 60 h in all samples. In the control SSF with no oligo-1,6-glucosidase, 5.4 g/L panose was detected after 60 h, which decreased to 3.8 g/L after 72 h (FIG. 8). In the SSFs containing Agl1, 2.7 g/L panose was detected after 60 h (50% reduction in panose compared to control) which was completely hydrolysed after 72 h. In the SSF containing Gth, 2.3 g/L panose was detected after 60 h (57% reduction in panose compared to control), which was completely hydrolysed after 72 h.

Conclusion:

The non-fermentable sugar panose was produced during SSF with commercial liquefied starch initially containing no non-fermentable sugars. Panose hydrolysis proceeded faster with the addition of oligo-1,6-glucosidase compared to the control containing no oligo-1,6-glucosidase and was completely hydrolysed to fermentable sugars during the process, leading to an overall reduction in DP3 residual sugars.

### Example 5

Biochemical Characterization

Activity Assay

For activity measurements, the enzyme Agl1 was incubated with 10 mM and 50 mM panose, isomaltose, isomaltotriose, maltose, maltotriose, and trehalose for 10 min at 37° C. in Britton-Robertson buffer, pH 5. The enzyme Gth was incubated with 15 mM panose, 10 and 50 mM isomaltose, and 10, 50 and 100 mM maltotriose and trehalose for 10 min at 37° C. in Britton-Robertson buffer, pH 5.

The reaction was stopped with 60 μL of 1 M NaOH, followed by addition of 60 μL of 1 M HCL.

The amount of produced glucose was quantified using a standardized glucose assay kit (D-Glucose HK Assay Kit, Megazyme u.c. IRE).

Glucose concentration was determined as specified in the instructions. Units are defined as the amount of enzyme required to produce one μmol of glucose per minute.

The pH range was investigated from pH 3 to pH 9 by adjusting the Britton-Robinson buffer to the respective pH.

The temperature range was investigated from 30° C.-80° C. by incubating the enzymes and measuring their activity at the respective temperature.

Temperature stability was determined by pre-incubating Agl1 at 50° C. and Gth at 80° C. and measuring the time required to reach 50% of the initial activity.

Process stability was determined by incubating the enzymes under process conditions (SSF medium without starch, pH 4.8, 30° C.) for 72 h and measuring the remaining activity.

Storage stability was determined by measuring the activity of the enzymes stored in a glass container in a stabilizing formulation (50% v/v glycerol) at 4° C. over a time period of 12 months. The storage stability was extrapolated from these data. “Without a loss in activity” is defined as an extrapolated activity of greater than 95% of the initial activity.

Product inhibition was determined by measuring hydrolysis of p-nitrophenyl-α-D-glucopyranoside (pNPG, Megazyme u.c., Ireland) by oligo-1,6-glucosidase activity in the presence of glucose or maltose from 0-50% (w/v). pNPG was quantified photometrically at 405 nm. Product inhibition is defined as IC50, which describes the amount of inhibitor required to reduce the enzyme activity by 50% at 2 mM pNPG.

Results:

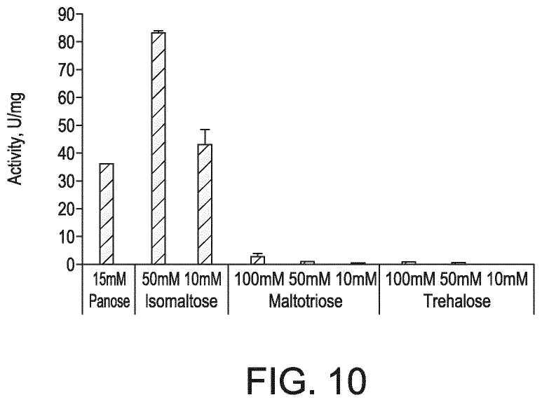

Activity measurements confirmed degradation of the oligosaccharides panose, isomaltose and isomaltotriose (FIG. 9, FIG. 10). Agl1 additionally breaks down trehalose. In the case of panose, one equivalent of glucose and one equivalent of maltose is produced. For isomaltose, isomaltotriose and trehalose, glucose is the sole product in two, three and two equivalents.

The pH range of Agl1 is 4.5-8.0, the temperature range is from 30-50° C. The pH range of Gth is 4.5-8.0, the temperature range is from 30-70° C.

Agl1 has a thermal stability of t½=30 min at 50° C. (time required for denaturation of 50% of the enzyme), a process stability of 80% after 72 h at 30° C. and a storage stability at 4° C. of 18-24 month without a loss in activity.

Gth has a thermal stability of t½=30 min at 80° C., a process stability of 80% after 72 h at 30° C. and a storage stability at 4° C. of 18-24 month without a loss in activity.

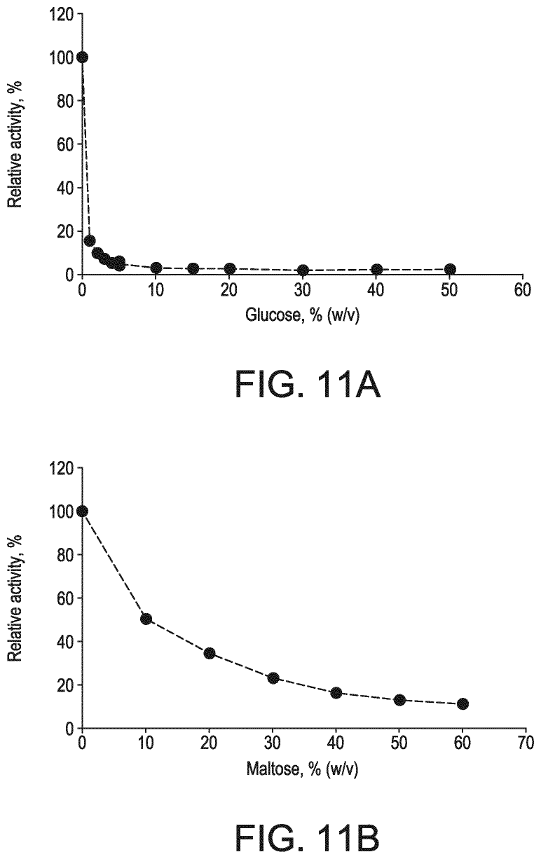

Product inhibition of Agl1 shows an IC50 for glucose of 0.6% w/v or 6 g/L and for maltose of 10% w/v or 100 g/L (FIG. 11A, B).

Conclusion:

The enzymes Agl1 and Gth have different optimal temperature ranges with regard to activity and stability. Agl1 is less stable and loses 50% activity after 30 min at 50° C., whereas Gth is very stable and showed 50% loss of activity after 30 min at 80° C. Both enzymes are stable for the whole process duration of 72 h at 30° C. This is important as the high glucose and maltose concentration in the beginning of the process inhibits the enzymes with oligo-1,6-glucosidase activity. Biochemical characterization shows different substrate spectra and different substrate specificity, which do not influence performance in the invention described above.

### Definitions

As used herein the term “substantially”, such as in “substantially consists”, will be understood by the person skilled in the art. The term “substantially” may also include embodiments with “entirely”, “completely”, “all”, etc. Hence, in embodiments the adjective substantially may also be removed. Where applicable, the term “substantially” may also relate to 90% or higher, such as 95% or higher, especially 99% or higher, even more especially 99.5% or higher, including 100%. The term “comprise” includes also embodiments wherein the term “comprises” means “consists of”. The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

The various aspects discussed in this patent can be combined in order to provide additional advantages. Furthermore, some of the features can form the basis for one or more divisional applications.

### CITED NON-PATENT DOCUMENTS

Zyprian & Matzura 1986: Zyprian, E., Matzura, H.: Characterization of signals promoting gene expression on the Staphylococcus aureus plasmid pUB110 and development of a gram-positive expression vector system. DNA 5: 919-25 (1986).
Dartois et al. 1994: Dartois, V., Copp6e, Y.-C., Colson, C, Baulard, A.: Genetic analysis and overexpression of lipolytic activity in Bacillus subtilis. Applied and Environmental Microbiology 60, 1670-1673 (1994).

### EMBODIMENTS

Amongst others, the invention provides embodiments, such as also described above, wherein the embodiments are only numbered for the sake of reference:

1. A process for the preparation of a fermentable sugar composition, wherein a polysaccharide composition is treated by at least one protein having glucoamylase enzymatic activity and at least one protein having oligo-1,6-glucosidase enzymatic activity, and wherein the fermentable sugars are removed during the treatment with the protein having oligo-1,6-glucosidase enzymatic activity.
2. The process according to embodiment 1, wherein the polysaccharide composition is treated by a mixture of at least one protein having glucoamylase enzymatic activity and at least one protein having oligo-1,6-glucosidase enzymatic activity.
3. The process according to embodiment 1, wherein the polysaccharide composition is first treated by at least one protein having glucoamylase enzymatic activity and the resulting composition is subsequently treated with at least one protein having oligo-1,6-glucosidase enzymatic activity.
4. The process according to any of the preceding embodiments, wherein the fermentable sugars are consumed in a fermentation process.
5. The process according to embodiment 4, wherein the fermentable sugar composition is fermented to yield an alcohol.
6. The process according to embodiment 4, wherein the fermentable sugar composition is fermented to yield an organic acid, such as citric acid, gluconic acid, lactic acid, acetic acid, L-ascorbic acid, itaconic acid, propionic acid, succinic acid and/or pyruvic acid, or an amino acid.
7. The process according to any of the preceding embodiments, wherein the polysaccharide composition is a starch hydrolysate.
8. The process according to any of the preceding embodiments, wherein the protein having glucoamylase enzymatic activity is a glucoamylase enzyme belonging to the enzyme class EC 3.2.1.3.
9. The process according to any of the preceding embodiments, wherein the protein having oligo-1,6-glucosidase enzymatic activity is an oligo-1,6-glucosidase enzyme belonging to the enzyme class EC 3.2.1.10.
10. The process according to any of the preceding embodiments, wherein a protein having oligo-1,6-glucosidase enzymatic activity derived from Bifidobacterium breve UCC2003 (Agl1) and/or derived from Parageobacillus thermoglucosidasius (Gth) is used.
11. The process according to embodiment 10, wherein the protein having oligo-1,6-glucosidase enzymatic activity is a protein having at least 50%, such as at least 55%, or at least 60%, or at least 65%, or at least 70%, or at least 75%, or at least 80%, or at least 85%, or at least 90%, or at least 95%, or at least 98% sequence identity to any of the oligo-1,6-glucosidase of Bifidobacterium breve UCC2003 (Agl1) or the oligo-1,6-glucosidase enzyme of Parageobacillus thermoglucosidasius (Gth).
12. The process according to any of the preceding embodiments, wherein the treatment by the enzyme mixture takes place in an aqueous process medium.
13. The process according to any of the preceding embodiments, wherein the treatment by the enzyme mixture takes place at an acidic pH in the range of 4.0 to 8.0, with an optimal range of pH 4.0 to 5.5, more specifically at pH 4.3 to 4.8.
14. The process according to any of the preceding embodiments, wherein the treatment by the enzyme mixture takes place at a temperature in the range of 30 to 70° C., with an optimal range of 30 to 50° C., more specifically of 30 to 35° C.
15. A process for the production of a fermentation product from a polysaccharide composition, wherein the polysaccharide composition is treated by at least one protein having glucoamylase enzymatic activity and at least one protein having oligo-1,6-glucosidase enzymatic activity, and wherein the resulting fermentable sugar composition is removed during the treatment with the protein having oligo-1,6-glucosidase enzymatic activity by a fermentation process in a fermentation medium, and wherein the desired fermentation product is isolated from the fermentation medium.
16. The process according to embodiment 15, wherein the fermentation product is selected from an alcohol, an organic acid, and an amino acid.
17. A process for the production of a fermentation product from a polysaccharide composition, characterized in that a polysaccharide composition is treated according to the process of embodiment 1 (or any of the other preceding embodiments 2-14), and subsequently a fermentation process is carried out in a fermentation medium, and wherein the desired fermentation product is isolated from the fermentation medium.
18. The process according to any of the preceding embodiments, such as embodiments 1-9 and/or embodiments 15-17, wherein a protein having oligo-1,6-glucosidase enzymatic activity from Bifidobacterium breve UCC2003 (Agl1) and/or from Parageobacillus thermoglucosidasius (Gth) is used.
19. The process according to embodiment 18, wherein the protein having oligo-1,6-glucosidase enzymatic activity is a protein having at least 50%, such as at least 55%, or at least 60%, or at least 65%, or at least 70%, or at least 75%, or at least 80%, or at least 85%, or at least 90%, or at least 95%, or at least 98% sequence identity to any of the oligo-1,6-glucosidase of Bifidobacterium breve UCC2003 (Agl1) represented by SEQ ID NO:1 or the oligo-1,6-glucosidase enzyme of Parageobacillus thermoglucosidasius (Gth) represented by SEQ ID NO:2.

The term “plurality” refers to two or more.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A process for the preparation of a fermentable sugar composition, wherein a polysaccharide composition is treated by at least one protein having glucoamylase enzymatic activity and at least one protein having oligo-1,6-glucosidase enzymatic activity, and wherein the fermentable sugars are removed during the treatment with the protein having oligo-1,6-glucosidase enzymatic activity.

2. The process according to claim 1, wherein the polysaccharide composition is treated by a mixture of at least one protein having glucoamylase enzymatic activity and at least one protein having oligo-1,6-glucosidase enzymatic activity.

3. The process according to claim 1, wherein the polysaccharide composition is first treated by at least one protein having glucoamylase enzymatic activity and the resulting composition is subsequently treated with at least one protein having oligo-1,6-glucosidase enzymatic activity.

4. The process according to claim 1, wherein the fermentable sugars are consumed in a fermentation process.

5. The process according to claim 4, wherein the fermentable sugar composition is fermented to yield an alcohol.

6. The process according to claim 4, wherein the fermentable sugar composition is fermented to yield an organic acid, such as citric acid, gluconic acid, lactic acid, acetic acid, L-ascorbic acid, itaconic acid, propionic acid, succinic acid and/or pyruvic acid, or an amino acid.

7. The process according to claim 1, wherein the polysaccharide composition is a starch hydrolysate.

8. The process according to claim 1, wherein the protein having glucoamylase enzymatic activity is a glucoamylase enzyme belonging to the enzyme class EC 3.2.1.3.

9. The process according to claim 1, wherein the protein having oligo-1,6-glucosidase enzymatic activity is an oligo-1,6-glucosidase enzyme belonging to the enzyme class EC 3.2.1.10.

10. The process according to claim 1, wherein a protein having oligo-1,6-glucosidase enzymatic activity from Bifidobacterium breve UCC2003 (Agi1) and/or from Parageobacillus thermoglucosidasius (Gth) is used.

11. The process according to claim 10, wherein the protein having oligo-1,6-glucosidase enzymatic activity is a protein having at least 50%, such as at least 55%, or at least 60%, or at least 65%, or at least 70%, or at least 75%, or at least 80%, or at least 85%, or at least 90%, or at least 95%, or at least 98% sequence identity to any of the oligo-1,6-glucosidase of Bifidobacterium breve UCC2003 (Agl1) represented by SEQ ID NO:1 or the oligo-1,6-glucosidase enzyme of Parageobacillus thermoglucosidasius (Gth) represented by SEQ ID NO:2.

12. The process according to claim 1, wherein the treatment by the enzyme mixture takes place in an aqueous process medium.

13. The process according to claim 1, wherein the treatment by the enzyme mixture takes place at an acidic pH in the range of 4.0 to 8.0, with an optimal range of pH 4.0 to 5.5, more specifically at pH 4.3 to 4.8.

14. The process according to claim 1, wherein the treatment by the enzyme mixture takes place at a temperature in the range of 30 to 70° C., with an optimal range of 30 to 50° C., more specifically of 30 to 35° C.

15. A process for the production of a fermentation product from a polysaccharide composition, characterized in that a polysaccharide composition is treated according to the process of claim 1, and subsequently a fermentation process is carried out in a fermentation medium, and wherein the desired fermentation product is isolated from the fermentation medium.

16. The process according to claim 15, wherein the fermentation product is selected from an alcohol, an organic acid, and an amino acid.


SEQUENCE LISTING (2 sequences)

SEQ ID NO: 1 - protein, 607 aa, Bifidobacterium breve UCC2003
  ACM89182.1 alpha-glucosidase 1
MMTTFNRAIIPDAIRTNGATPNPWWSNAVVYQIYPRSFQDTNGDGFGDLKGITSRLDYLADLGVDVLWLSPVYKSPQDDNGYDISDYQDIDPLFGTLDDMDELLAEAHKRGLKVVMDLVVNHTSDEHAWFEASKNKDDEHADWYWWRPARPGTTPGEPGSEPNQWGSYFGGSAWEYCPERGEYYLHQFSKKQPDLNWENPAVRRAVYDMMNWWLDRGIDGFRMDVITLISKRTDANGRLPGEYGSELDDLPVGEEGYSNPNPFCADGPRQDEFLKEMRREVFAGREGFLTVGEAPGITPVRNEHITNPANGELDMLFLFDHVDFDCDGVKWKPLPLDLPGFKRIMAGYQTAVENVGWASLFTGNHDQPRVVSRWGDDSSEESRVRSAKALGLMLHMHRGTPYVYQGEELGMTNAHFTSLDQYRDLESLNAYRQRVEEAKVQSPESMLAGIAARGRDNSRTPMQWDGSAYAGFTAPDAATEPWISVNPNHAEINAAGEFDDPDSVYAFYKKLIALRHNSSIVAAGEWQLIDADDAHVYAFTRTLGNERLLVVVNLSGRTVDLPRESTELIAGGVTEPDIILSTYDAPHTVVSLANRELDPWEAAAVQL

SEQ ID NO: 2 - protein, 562 aa, Parageobacillus thermoglucosidasius
  WP_042386239.1 alpha-glucosidase
MERVWWKEAVVYQIYPRSFYDSNGDGIGDIRGIIAKLDYLKELGVDVVWLSPVYKSPNDDNGYDISDYRDIMDEFGTMADWKTMLEEMHKRGIKLVMDLVVNHTSDEHPWFIESRKSKDNPYRDYYIWRPGKNGKEPNNWESVFSGSAWEYDEMTGEYYLHLFSKKQPDLNWENPKVRREVYEMMKFWLDKGVDGFRMDVINMISKVPELPDGEPQSGKKYASGSRYYMNGPRVHEFLQEMNREVLSKYDIMTVGETPGVTPKEGILYTDPSRRELNMVFQFEHMDLDSGPGGKWDIRPWSLADLKKTMTKWQKELEGKGWNSLYLNNHDQPRAVSRFGDDGKYRVESAKMLATFLHMMQGTPYIYQGEEIGMTNVRFPSIEDYRDIETLNMYKERVEEYGEDPQEVMEKIYYKGRDNARTPMQWDDSENAGFTAGTPWIPVNPNYKEINVKAALEDPNSVFHYYKKLIQLRKQHDIIVYGTYDLILEDDPYIYAYTRTLGNEQLIVITNFSEKTPVFRLPDHIIYKTKELLISNYDVDEAEELKEIRLRPWEARVYKIRLP